(19) United States
(12) Patent Application Publication
(10) Pub. No.: US 2019/0180866 A1
(43) Pub. Date: Jun. 13, 2019

(54) APPARATUS AND METHOD FOR OPTIMIZING THE TIME ALLOCATION OF A CLINICAL PROCESS

(71) Applicant: KONINKLIJKE PHILIPS N.V., Eindhoven (NL)

(72) Inventors: Tak Ming Chen, Shanghai (CN); Choo Chiap Chiau, Shanghai (CN); Qizhong Lin, Shanghai (CN); Johanna Maria De Bont, Eindhoven (NL); Niels Laute, Eindhoven (NL); Jurriën Carl Gosselink, Lichtenvoorde (NL)

(21) Appl. No.: 14/810,829

(22) Filed: Jul. 28, 2015

(30) Foreign Application Priority Data
Oct. 30, 2014 (EP) 14191053

(51) Int. Cl. G16H 40/20; G06Q 10/06

(52) U.S. Cl. CPC G16H 40/20; G16H 80/00; G06Q 10/0631

(57) ABSTRACT

The present invention generally relates to time allocation of a clinical process. It is proposed in the present invention to introduce statistical significance as an indicator of the real improvement of activities in the modified time allocation against the original time allocation to achieve a statistically meaningful time allocation.

### CROSS-REFERENCE TO PRIOR APPLICATIONS

This application claims the benefit or priority of and describes relationships between the following applications: PCT International Application No. PCT/CN2014/085554, filed on Aug. 29, 2014 and European Patent Application No. 14191053.9, filed on Oct. 30, 2014, both of which are incorporated herein in whole by reference.

### FIELD OF THE INVENTION

The present invention generally relates to time allocation of a clinical process.

### BACKGROUND OF THE INVENTION

In clinical processes, time allocation is critical for saving lives and improving operations. For example in the case of a patient arriving at the emergency department (ED) who requires percutaneous coronary intervention (PCI) for a ST-Elevation Myocardial Infarction (STEMI), commonly known as heart attack, the mortality rate will be significantly increased if it takes more than 90 minutes from the patient's arrival at the emergency department (door) to completion of the treatment (balloon placed). Therefore, allocation the elapsed time, such as the Door-to-Balloon (D2B) time, to improve the duration is of life-critical importance or and is also wise in terms of performance.

The availability of a patient population's time data for a clinical process (e.g. D2B of the STEMI protocol) implies that automatic methods are available to report the basic statistics for the process time as described in patent US20120123803 [1]. These reporting functions enable the users to look into different groups' process times manually. However, they provide nothing further for investigating a solution for time allocation to reach a specific target duration with reference to sound statistical characteristics, nor do they offer an optimized statistically relevant solution to reach a specific target duration given the current time allocation. In this way, an allocation is generated for a target duration regardless of its statistical characteristics, which may result in in a waste of allocation efforts if the generated time allocation is applied. Therefore, it is desired to have a method or apparatus by means of which the generation of statistically meaningless target allocations without real improvement is avoided.

### SUMMARY OF THE INVENTION

To achieve this, it would be desirable to take statistical and quantitative measurement of improvement into account for time allocation of a clinical process.

To better address one or more of these concerns, according to an embodiment of one aspect of the invention, an apparatus and method for time allocation of a clinical process is provided.

An apparatus 500 for optimizing the time allocation of a clinical process, the clinical process comprising a plurality of consecutive activities, the apparatus comprising:

a receiving unit 501 adapted for receiving an original time allocation for the clinical process, the original time allocation comprising an original duration for each activity;

a generating unit 502 adapted for generating a reference time allocation for the clinical process, the reference time allocation comprising a reference duration for each activity, the reference duration being calculated based on a statistical significance threshold set for each activity and a predefined statistical significance evaluation model of each activity, wherein the predefined statistical significance evaluation model of the activity indicating the relation between the statistical significance and the duration of the activity is built on historical time allocation records;

wherein the statistical significance evaluation model is formulated by statistical comparisons between multiple durations for an activity in historical time allocation records and the multiple durations adjust, the adjustment traversing differences between a duration of the activity in a feasible time allocation and the average duration of the activity in historical time allocation records, the statistical comparison result determining the statistical significance value of the duration of the activity in the feasible time allocation.

The clinical process is decomposed into a plurality of consecutive activities.

It is proposed in the present invention to introduce statistical significance as an indicator of the real improvement of activities in the modified time allocation against the original time allocation for statistically meaningful time allocation. For example, assume that the duration of an activity “Transfer” in D2B in history records is very unlikely (p=0.02) to have a random subset of records that is 5% shorter than the overall duration. And the duration of an activity “Patient Consent” can easily have a random subset (p=0.2) with 5% shorter duration than the overall. Then an improvement of 5% duration for the activity “Transfer” is more statistically meaningful than 5% for the activity “Patient Consent”, since the 5% improvement in “Transfer” will more likely reflect real efforts rather than pure luck; while one can observe 5% improvement simply by chance in “Patient Consent”, without no efforts spent. More specifically, a statistical significance evaluation model is established based on historical records of each activity to investigate the relation between a certain adjustment of an activity duration and the corresponding statistical significance based on historical records, where the statistical significance value serves as an indicator for the non-trivialness of duration adjustment of the activity. Given an original duration and a statistical significance threshold of each activity, a reference duration of each activity is determined, thus generating a reference time allocation comprising multiple reference durations. The reference duration provides the statistical information for the user for further modification. In this way, the further modification of the time allocation for a clinical process is more practically efficient history to take historical information into account and focus the limited sources to make real improvement.

In an embodiment, apparatus further comprises a determining unit 503 adapted for determining a priority of each activity for modification, based on the quantitative deviation between the original duration and the reference duration for the activity.

The activities can be prioritized for modification based on quantitative deviation (e.g. difference) between the original activity duration and the reference duration. Thus a detailed instruction is given to the user to help the user to make use of the available information.

In an embodiment, the apparatus further comprises

an optimizing unit 504 adapted for modifying the original time allocation to generate an optimized time allocation to achieve a target duration of the clinical process,

wherein the value of the original duration is modified towards the value of the reference duration for each activity based on the determined priority;

wherein the modified original time allocation achieving the target duration of the clinical process is the optimized time allocation.

The original durations are modified one by one based on the determined priorities until the overall duration reaches the target duration of the whole clinical process. However, when the target duration of the whole clinical process has not been achieved, the value of an original duration is modified gradually to the value of the corresponding reference duration until the modified original duration of an activity equals to the corresponding reference duration, and next the prioritized original duration of another activity is further modified to achieve the target duration of the whole clinical process. The value of a modified original duration of an activity can be any value between the value of the original duration and the value of the corresponding reference duration. The modified original time allocation satisfying the requirement of the target duration of the clinical process is determined as the optimized time allocation, which is statistically meaningful. The optimized time allocation is more practical and efficient.

In one embodiment, either the activity comprising a larger quantitative deviation from the original duration possesses higher priority or the activity comprising a smaller quantitative deviation from the original duration possesses higher priority.

The priorities can be determined by different rules for different purposes. If the purpose is to minimize the number of activities modified in order to concentrate the limited allocation efforts for the most effective improvements, the activity with a larger modification difference possesses higher priority. If the purpose is to maximize the realization of the activities which are statistically meaningful for the overall generated time allocation, the activity with a smaller modification difference possesses higher priority. The rule can also be defined by a user according to other considerations by weighting.

In one embodiment, the target duration of the clinical process is obtained based on a target statistical significance set for the clinical process and a predefined statistical significance evaluation model of the clinical process, wherein the predefined statistical significance evaluation model of the clinical process indicating the relation between the statistical significance and the duration of the clinical process allocation is built on historical time allocation records; wherein the statistical significance evaluation model is formulated by statistical comparisons between multiple durations for the clinical process in historical time allocation records and the multiple durations adjusted, the adjustment traversing differences between a duration of the clinical process in a feasible time allocation and the average duration of the clinical process in historical time allocation records, the statistical comparison result determining the statistical significance value of the duration of the clinical process in the feasible time allocation.

As an alternative, a target statistical significance can also be set for the clinical process, which can be converted to a target duration, based on the target statistical significance and a predefined statistical significance evaluation model of the clinical process based on historical time allocation records.

In one embodiment, the quantitative deviation is the difference, variance or time proportion modification, expressed in a percentage figure, between the original duration and the reference duration.

The quantitative measurement between the original duration and the reference duration can be targeted on the difference, variance or time proportion modification, expressed in a percentage figure. Therefore, different observations of statistic characteristics of the time allocation are provided.

In one embodiment, the original duration of the activity executed in real time is excluded from the original allocation and reference allocation.

The apparatus is applicable to the real time situation. The optimized time allocation of the remaining unexecuted activities is provided during the conducting of the original time allocation, thus providing a user with instant guidance for the duration allocation for each remaining activity.

In one embodiment, the apparatus further comprises a visualizing unit 505 adapted for visualizing a time allocation, wherein a sequence comprising non-overlapped consecutive duration indicators for the activities is presented aligned with a duration indicator for the clinical process at both ends; wherein each duration indicator represents the duration of one activity or the clinical process, the length of the duration indicator correlated with the duration of the activity or the clinical process represented. The duration value and the statistical significance value of the activity or the clinical process are displayed.

The visualizing unit 505 gives an explicit visualization of a time allocation, in which the duration of the clinical process and the durations of the activities are presented aligned to offer a simple and clear view of the relations between the duration of the clinical process and the durations of the decomposed activities thereof. All durations and statistical information may be further displayed, providing a convenient interface for instantaneous adjusting.

In one embodiment, a visualizing unit 504 is adapted further for visualization of the intervention of the time allocation:

moving one end of a duration indicator for an activity or the clinical process;

aligning duration indicators for the activities, which indicators are to be connected consecutively; aligning the sequence comprising consecutively connected duration indicators with the duration indicator of the clinical process at both ends.

In one embodiment, a visualizing unit 504 is adapted further for visualization of intervention in the time allocation:

moving one end of a duration indicator for an activity of the clinical process;

updating the duration value displayed and the statistical significance value displayed of the activity, based on the changed duration and the predefined statistical significance model of the activity;

updating the duration value displayed and the statistical significance value displayed of the clinical process, based on the changed duration and the predefined statistical significance model of the clinical process;

aligning duration indicators for the activities, which indicators are to be connected consecutively; aligning the sequence comprising consecutively connected duration indicators with the duration indicator of the clinical process at both ends.

Besides the illustration of the time allocation, interactive adjusting is also enabled according to user preference, which further offers customization of time allocation. The intervention of the user is presented on the interface to the related duration indicator, while other indicators and values are updated simultaneously to give a general view of the change.

In one embodiment, a visualizing unit 504 is adapted further for visualization of prioritization of activities, wherein the indicator with highest priority is displayed so as to be distinguishable from other indicators by providing the indicator with a distinguishing visual property. The distinguishing visual property can be a distinguishing color, remark or movement of the indicator.

The invention comprises a method for time allocation of a clinical process, the clinical process comprising a plurality of consecutive activities, and the method comprising:

receiving an original time allocation for the clinical process, the original time allocation comprising an original duration for each activity;

generating a reference time allocation for the clinical process, the reference time allocation comprising a reference duration for each activity, the reference duration being calculated based on a statistical significance threshold set for each activity and a predefined statistical significance evaluation model of each activity, wherein the predefined statistical significance evaluation model of the activity indicating the relation between the statistical significance and the duration of the activity is built on historical time allocation records;

determining a priority of each activity, based on the difference between the original duration and the reference duration for the activity;

wherein the statistical significance evaluation model is formulated by a statistical comparison between multiple durations for an activity in historical time allocation records and the multiple durations adjusted, the adjustment traversing differences between a duration of the activity in a feasible time allocation and the average duration of the activity in historical time allocation records, the statistical comparison result determining the statistical significance value of the duration of the activity in the feasible time allocation.

Various aspects and features of the disclosure are described in further detail below. And other objects and advantages of the present invention will become more apparent from the following description when taken in conjunction with the accompanying drawings.

### DESCRIPTION OF THE DRAWINGS

The present invention will be described and explained hereinafter in more detail in combination with embodiments and with reference to the drawings, wherein:

FIG. 1 is a schematic diagram showing a flowchart of a time allocation method in an embodiment of the invention.

FIG. 2 is a schematic diagram showing the decomposing of a clinical process in an embodiment of the invention.

FIG. 3(a) is a schematic diagram showing historical time allocation records in an embodiment of the invention.

FIG. 3(b) is a schematic diagram showing the activity 202 in historical time allocation records of FIG. 3(a).

FIG. 3(c) is a schematic diagram showing the adjusted activity 202 of FIG. 3(a).

FIG. 4(a) is a schematic diagram showing a user interface in an embodiment of the invention.

FIG. 4 (b) is a schematic diagram showing a user interface illustrating a time allocation after user intervention in an embodiment of the invention.

FIG. 5 is a schematic diagram showing the time allocation apparatus in an embodiment of the invention.

The same reference signs in the drawings indicate similar or corresponding features and/or functionalities.

### DETAILED DESCRIPTION

The present invention will be described with respect to particular embodiments and with reference to certain drawings, but the invention is not limited thereto but only by the claims. The drawings described are only schematic and are non-limiting. In the drawings, the size of some of the elements may be exaggerated and not drawn to scale for illustrative purposes.

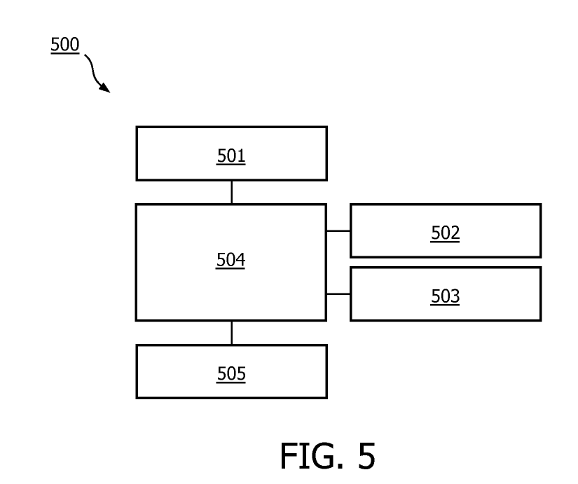

The invention proposes an apparatus for time allocation of a clinical process based on historical records. FIG. 5 is a schematic diagram showing the time allocation apparatus in an embodiment of the invention. The clinical process is decomposed into multiple activities as a prerequisite for time allocation. FIG. 2 is a schematic diagram showing the decomposing of a clinical process in an embodiment of the invention. The clinical process 200 is decomposed into sequential activities referred to as activity 201 to activity N. The decomposition can be based on medical guidelines, requirements for data repository organization or user interest. The clinical process can be any process defined in medical guidelines, where time is a key performance indicator, including but not limited to cases of heart attack, stroke, choking, bleeding, injuries, etc. For example, a D2B clinical process can be decomposed into six consecutive activities referred to as: Door-to-Activation, Physician Arrival, Patient Consent, Paper Work, Transfer and Operation to Balloon. Then a target duration is set for the clinical process and at least one statistical significance threshold is set for a plurality of activities, based on user interest or some medical guideline. As for statistical thresholds, they can be implemented as one overall statistical significance threshold for all desired activities. A statistical threshold value that is generally accepted as statistically significant is 0.05 (http://en.wikipedia.org/wiki/Statistical_significance). Optionally, it can be implemented as several statistical significance thresholds of different values allocated to a respective number of activities. For example, a lower statistical significance threshold can be assigned to an essential activity to have a comparable assurance of real improvement. As an alternative, the target of the clinical process can also be a target statistical significance instead of a target duration. The target statistical significance can be converted by the statistical significance evaluation model of the clinical process to a target duration. The setting of a target duration or, alternatively, a statistical significance value of the clinical process and a statistical significance threshold of each activity can be predefined or manually input by a user. Receiving unit 501 receives an original time allocation. The original time allocation can be an averaged time allocation based on historical time allocation records, a time allocation currently being used or a time allocation recommended by a guideline as a basis for further statistical optimization. Generating unit 502 generates an optimized time allocation, based on a target duration of the clinical process, which is usually shorter than the overall duration of the original allocation, and at least a statistical significance threshold for a plurality of activities. The statistical significance evaluation model of an activity is established based on the activity's historical values in historical time allocations to estimate the statistical significance of the duration deviating from the historical basis. Given the significance threshold set for the activity as the input of the statistical significance evaluation model, a reference duration of the activity is calculated to achieve the significance threshold. The allocation of the generated reference duration enables the activity to achieve the significance threshold, which is either set by a user or predefined. After all estimations for reference durations of the activities are completed, the priorities of the activities for modifications are determined based on the duration modification needed against the original time allocation to achieve the reference duration. The activities are prioritized to be modified towards the value of the activity reference duration one by one to achieve the statistical significance threshold for each activity until the overall duration of the clinical process equals the target duration, thus generating a statistically optimized time allocation with the overall duration equal to the target duration of the clinical process. The activities can be prioritized based on other quantitative deviations for modification.

In an embodiment for determining the priorities, different rules can be set for comparison. In order to minimize the number of modified activities to concentrate the limited allocation efforts on the most effective improvements, the activity causing a larger modification or a greater time proportion modification, expressed in a percentage figure, between the original duration and the reference duration modification possesses higher priority. In order to maximize the realization of statistically meaningful activities for the overall generated time allocation, the activity causing a smaller modification or a smaller time proportion modification, expressed in a percentage figure, between the original duration and the reference duration modification possesses higher priority. The rule can also be defined by a user according to other considerations.

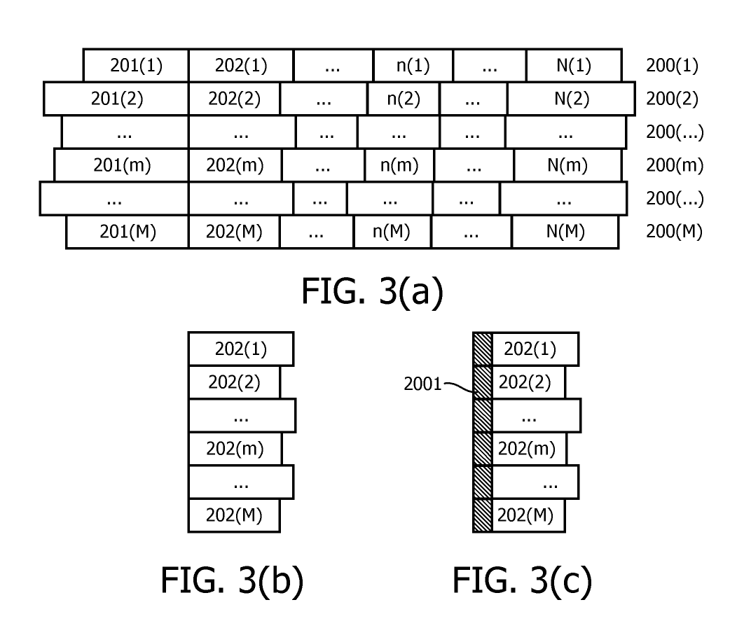

In an embodiment of the statistical significance evaluation model, the model is formulated by a statistical comparison of an activity for durations in historical time allocation records and in historical records adjusted as a possible optimized time allocation. FIG. 3(a) is an exemplification of M historical time allocation records, in which each row represents one time allocation executed previously. As illustrated in FIG. 3(a), each block n(m) indicates an activity, while the length of each block indicates the duration of the activity. By way of example, activity 202 is selected for further discussions about the statistical significance model establishment, which also applies to other activities. From the historical time allocation records, all durations executed once for activity 202 can be extracted, as illustrated in FIG. 3(b). These M durations including 202(1) to 202(M) constitute a sample for activity 202. A new sample is generated, wherein the old sample is subjected to an adjustment 2001 (shadowed) as illustrated in FIG. 3(c), where the adjustment 2001 is deducted from each duration of 202(1) to 202(M). The adjustment 2001 here is the difference between the activity duration in a possible time allocation and the averaged activity duration, wherein the averaged activity duration is the average duration value of 202(1) to 202(M). Then, a statistical comparison test is applied to these two samples to calculate the statistical significance as a statistical evaluation of the adjustment in a possible time allocation. When the significance is strong (e.g. the smaller the p-value, the more statistically significant the result), the activity duration to be evaluated in the possible target time allocation is statistically meaningful, which indicates that the proposed activity duration is more likely to be an actually improved state than a fluke. When the significance is weak, the activity duration to be evaluated in the possible time allocation is not statistically meaningful, which indicates that the proposed activity duration is more of a fluke, which is unlikely to mean real improvement. Such a computation can traverse all possible values of the activity durations, thus resulting in the significance evaluation model for activity 202, which reveals the relation between the duration of the activity and its corresponding significance. The possible values of the duration of the activity 202 can be limited to the scale of minimum and maximum duration values of the historical records and the interval between the possible values (eg. 1 minute) can be set to reach a reasonable computational complexity.

The comparison can take the form of statistical tests such as non-parametric tests: e.g. Wilcoxon rank-sum test, Mann-Whitney U test, Kolmogorov-Smirnov test, or parametric tests: e.g. student t-test (for normal distributions), according to different distributions of the activity, where the statistical measurement is statistical significance represented as p values. The computational method is also applied to the overall duration of the clinical process to get a statistical significance evaluation model of the clinical process. It is possible that multiple qualified configurations can be obtained for the optimized time allocation.

In one embodiment, the apparatus is applied to time allocation in real time. While the clinical process is being executed based on the original time allocation (e.g. being currently used), the original duration of the activity executed is sensed in real time by measuring equipment. Those corresponding activities already happened are excluded from the clinical process. Accordingly, the duration of the activity executed is excluded from the original allocation and the reference allocation. Then, an optimized time allocation of the updated clinical process comprising the remaining activities is generated by the apparatus mentioned above.

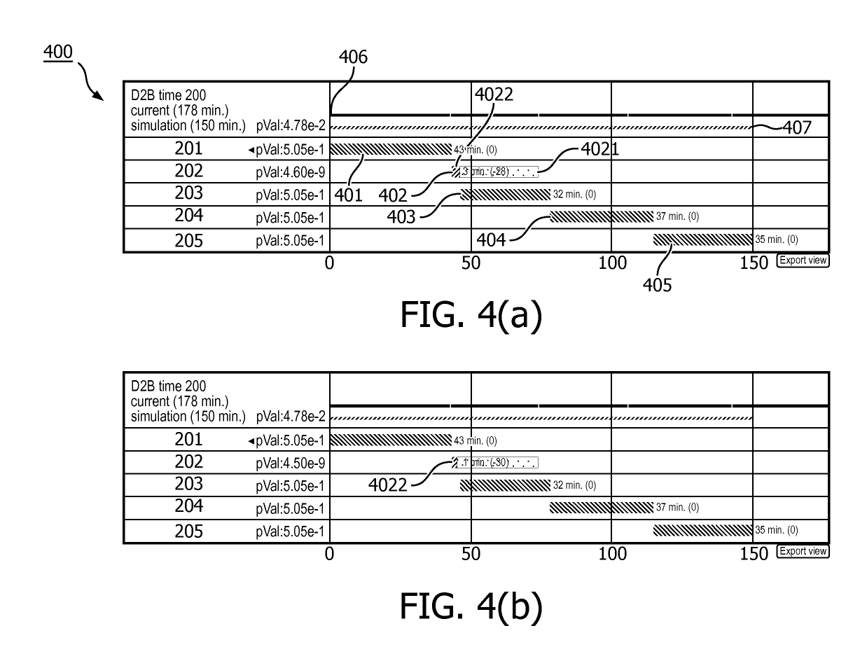

FIG. 4(a), FIG. 4(b) are embodiments of a user interface 400 of the optimized time allocation method of the invention shown as the output of the visualization unit 505, where the p-value represents the statistical significance value. The user interface 400 can illustrate a time allocation. As illustrated in FIG. 4 (a), the current time allocation, corresponding to a time allocation for a clinical process consisting of activities 201˜205, is represented by appropriate duration indicators, the length of which is proportional to the duration of the activity or the clinical process, while the duration value and the corresponding statistical significance value are displayed. The duration indicator 407, shown as a thin bar, is used to indicate the duration of the whole clinical process. Beneath it there are non-overlapped duration indicators 401˜405, shown as thick bars, to indicate durations for activities 201˜205, respectively. The duration indicators 401˜405 are consecutively connected end to end and constitute a sequence parallel to the clinical process duration indicator 407. The sequence comprising duration indicators 401˜405 is aligned with clinical process duration indicator 407 at both ends. The duration indicators 401˜405 are arranged in parallel in order to clearly show the duration value and the p-value. As an alternative, the duration indicators 401˜405 can be placed in one line.

An optional duration indicator 406 is arranged at the top to indicate the duration of the original time allocation for comparison, making it easy for the user to see the difference between the original time allocation and the optimized time allocation. An optional shadow block, as the original duration, is set so as to demonstrate an overlap with the duration indicator 402 to indicate the reduction between the left end 4021 and right end 4022. The duration value (3 minutes) in the target time allocation, the modification difference (−28 minutes) and the statistical significance of the modified activity (4.50e-9) are also displayed, wherein the position of related values (e.g. the statistical significance value, the modification difference value, duration value etc.) in FIG. 4 (a) should not been seen as a limitation to the embodiments.

The user interface 400 can also facilitate a new setting of the targets to update the generated allocation and the user intervention, which only changes based on a user's instruction due to possible actual limitation or preference. The interface 400 can further include input and display windows (not illustrated in FIG. 4(a), FIG. 4(b)) for setting a target duration or an alternative target statistical significance of the clinical process or a statistical significance threshold, while the conversion function can be deployed to display the converted statistical significance value of the target duration and the converted duration value of the target statistical significance, based on appropriate statistical significance models. After a new target or a threshold is set, an optimized time allocation will be automatically generated and presented on the user interface 400.

A manual adjustment of the duration indicator of the clinical process can be taken to be a new setting of the target duration of the clinical process. For example, the right end of the clinical process duration indicator 407 is moved to the left. The change of the indicator 407 can be interpreted as a change of duration based on the predefined relation between the length of the indicator and the duration the indicator represents. Then, the new duration target of the clinical process is set. An updated optimized time allocation will be presented on the user interface 400.

A manual adjustment of the duration indicator of a particular activity can be taken to be either a new setting of the statistical significance threshold or a user intervention. If the adjustment is taken to be a new setting of the statistical significance threshold, a new statistical significance is obtained based on the new duration due to the adjustment and the predefined statistical significance evaluation model. If the adjustment is taken to be a user intervention, only the duration of the activity will be changed and the duration of the clinical process is updated accordingly regardless of the target duration, while the other activities keep the previous status. As for FIG. 4(a), the user intervention can be implemented by moving one end of an activity duration indicator, such as the right end 4021, further to the left, while the indicator 403˜405 moves to the left accordingly to make sure that the sequence of activity duration indicators are consecutively interconnected end to end. All the related modification differences and the statistical significance will be updated simultaneously, based on the distance moved that is indicative of the duration change and the statistical significance evaluation models of the activity and the clinical process. The duration indicator 407 will shrink to the left to get aligned with the activities at the left end of indicator 401 and the right end of 405 as a consequence of the modification of the time allocation. FIG. 4(b) is an exemplary generated allocation after a user intervention causing the duration of activity 202 to be reduced to 1 minute, while the duration of the whole clinical process is reduced to 148 minutes. The distinction between a new setting and a user intervention can be made by means of an intervention button (not illustrated in FIG. 4(a), FIG. 4(b)) used as a flag input to the internal computation. A User intervention in an activity can also be a lengthening of the duration indicator, where the process remains the same except that the direction of movement is to the right. It applies to a situation in which a user may sacrifice the optimization of the statistical significance of some activity to secure some other activity that is more important due to guidelines or a realistic situation.

Optionally, in the case of user intervention, the prioritizations of the other activities, i.e. the non-intervened activities, are still calculated internally for modification support. Priorities are displayed as a suggestion to a user by using color, text, movement and other visible characteristics of the respective indicators. Implementation thereof can be achieved by visualizing only one indicator with highest priority or more indicators, as defined.

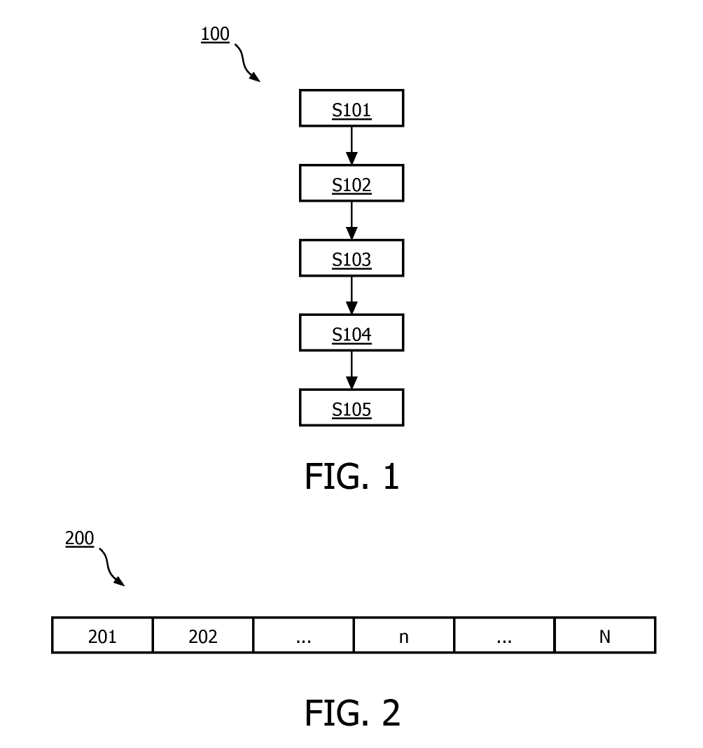

FIG. 1 is a schematic diagram showing the clinical process comprising a plurality of consecutive activities, the method 100 comprising the following steps:

Step S101, which includes receiving an original time allocation for the clinical process, the original time allocation comprising an original duration for each activity;

Step S102, which includes generating a reference time allocation for the clinical process, the reference time allocation comprising a reference duration for each activity, the reference duration being calculated based on a statistical significance threshold set for each activity and a predefined statistical significance evaluation model of each activity, wherein the predefined statistical significance evaluation model of the activity indicating the relation between the statistical significance and the duration of the activity is built on historical time allocation records;

Step S103, which includes determining a priority of each activity, based on the quantitative deviation between the original duration and the reference duration for the activity;

Step S104, which includes optimizing the original time allocation, based on a target duration of the clinical process and the determined priority of each activity, to generate an optimized time allocation, wherein the value of the original duration is modified towards the value of the reference duration for each activity, based on the determined priority for the modified original time allocation, to achieve the target duration of the clinical process, the modified original time allocation achieving the target duration of the clinical process being the optimized time allocation.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. An apparatus (500) for optimizing the time allocation of a clinical process, the clinical process comprising a plurality of consecutive activities, the apparatus comprising:
a receiving unit (501) adapted for receiving an original time allocation for the clinical process, the original time allocation comprising an original duration for each activity;
a generating unit (502) adapted for generating a reference time allocation for the clinical process, the reference time allocation comprising a reference duration for each activity, the reference duration being calculated based on a statistical significance threshold set for each activity and a predefined statistical significance evaluation model of each activity, wherein the predefined statistical significance evaluation model of the activity indicating the relation between the statistical significance and the duration of the activity is built on historical time allocation records;
wherein the statistical significance evaluation model is formulated by statistical comparisons between multiple durations for an activity in historical time allocation records and the multiple durations adjusted, the adjustment traversing differences between a duration of the activity in a feasible time allocation and the average duration of the activity in historical time allocation records, the statistical comparison result determining the statistical significance value of the duration of the activity in the feasible time allocation.

2. An apparatus (500) according to claim 1, the apparatus further comprising a determining unit (503) adapted for determining a priority of each activity for modification, based on the quantitative deviation between the original duration and the reference duration for the activity.

3. An apparatus (500) according to claim 1, the apparatus further comprising
an optimizing unit (504) adapted for modifying the original time allocation to generate an optimized time allocation to achieve a target duration of the clinical process,
wherein the value of the original duration is modified towards the value of the reference duration for each activity based on the determined priority;
wherein the modified original time allocation achieving the target duration of the clinical process is the optimized time allocation.

4. An apparatus (500) according to claim 3, wherein the target duration of the clinical process is obtained based on a target statistical significance set for the clinical process and a predefined statistical significance evaluation model of the clinical process,
wherein the predefined statistical significance evaluation model of the clinical process indicating the relation between the statistical significance and the duration of the clinical process allocation records is built on historical time allocation records;
wherein the statistical significance evaluation model is formulated by statistical comparisons between multiple durations for the clinical process in historical time allocation records and the multiple durations adjusted, the adjustment traversing differences between a duration of the clinical process in a feasible time allocation and the average duration of the clinical process in historical time allocation records, the statistical comparison result determining the statistical significance value of the duration of the clinical process in the feasible time allocation.

5. An apparatus (500) according to any one of claim 1, wherein the original duration of the activity executed in real time is excluded from the original allocation and reference allocation.

6. An apparatus (500) according to any one of claims 1-3, further comprising a visualizing unit (504) adapted for visualizing a time allocation, wherein a sequence comprising non-overlapped consecutive duration indicators for the activities is presented aligned with a duration indicator for the clinical process at both ends; wherein each duration indicator represents the duration of an activity or the clinical process, the length of the duration indicator correlated with the duration of the activity or the clinical process represented; visualizing a statistical significance corresponding to the duration of the activity or the time allocation.

7. An apparatus (500) according to claim 6, wherein the visualizing unit (504) adapted for visualizing the priority of the activity for modification by configuring the visual property of the corresponding duration indicator.

8. A method of optimizing the time allocation of a clinical process, the clinical process comprising a plurality of consecutive activities, the method comprising the steps of:
receiving (S101) an original time allocation for the clinical process, the original time allocation comprising an original duration for each activity;
generating (S202) a reference time allocation for the clinical process, the reference time allocation comprising a reference duration for each activity, the reference duration being calculated based on a statistical significance threshold set for each activity and a predefined statistical significance evaluation model of each activity, wherein the predefined statistical significance evaluation model of the activity indicating the relation between the statistical significance and the duration of the activity is built on historical time allocation records;
wherein the statistical significance evaluation model is formulated by statistical comparisons between multiple durations for an activity in historical time allocation records and the multiple durations adjusted, the adjustment traversing differences between a duration of the activity in a feasible time allocation and the average duration of the activity in historical time allocation records, the statistical comparison result determining the statistical significance value of the duration of the activity in the feasible time allocation.

9. A method according to claim 8, the method further comprising a step of determining (S503) a priority of each activity for modification, based on the quantitative deviation between the original duration and the reference duration for the activity.

10. A method according to claim 8, the apparatus further comprising
modifying the original time allocation to generate an optimized time allocation to achieve a target duration of the clinical process,
wherein the value of the original duration is modified towards the value of the reference duration for each activity based on the determined priority;
wherein the modified original time allocation achieving the target duration of the clinical process is the optimized time allocation.

11. An method according to any one of claim 10, wherein the target duration of the clinical process is obtained based on a target statistical significance set for the clinical process and a predefined statistical significance evaluation model of the clinical process,
wherein the predefined statistical significance evaluation model of the clinical process indicating the relation between the statistical significance and the duration of the clinical process allocation records is built on historical time allocation records;
wherein the statistical significance evaluation model is formulated by statistical comparisons between multiple durations for the clinical process in historical time allocation records and the multiple durations adjusted, the adjustment traversing differences between a duration of the clinical process in a feasible time allocation and the average duration of the clinical process in historical time allocation records, the statistical comparison result determining the statistical significance value of the duration of the clinical process in the feasible time allocation.

12. A method according to any one of claim 8, wherein the original duration of the activity executed in real time is excluded from the original allocation and reference allocation.

13. A method according to any one of claims 8-11, further comprising a step of visualizing (S105) a time allocation, wherein a sequence comprising non-overlapped consecutive duration indicators for the activities is presented aligned with a duration indicator for the clinical process at both ends; wherein each duration indicator represents the duration of an activity or the clinical process, the length of the duration indicator correlated with the duration of the activity or the clinical process represented; further comprising a step of visualizing a statistical significance corresponding to the duration of the activity or the time allocation.

14. A method according to any one of claim 13, further comprising a step of visualizing the priority of the activity for modification by configuring the visual property of the corresponding duration indicator

15. A computer program product comprising computer program code means for causing a computer to perform the steps of the method as claimed in claim 8 when said computer program code means is run on the computer.
